(19) United States
(12) Patent Application Publication
(10) Pub. No.: US 2022/0380715 A1
(43) Pub. Date: Dec. 1, 2022

(54) PRODUCTION AND USE OF COMPACTED COLLAGEN

(71) Applicant: 3D Spine Matrix Biotechnologie GmbH, Krems/Donau (AT)

(72) Inventors: Christian Burgard, St. Wendel (DE); Alex Krabler (Deceased), Muehldorf (AT)

(73) Assignee: 3D Spine Matrix Biotechnologie GmbH, Krems/Donau (AT)

(21) Appl. No.: 17/773,981

(22) PCT Filed: Nov. 4, 2020
(86) PCT No.: PCT/AT2020/060387

§ 371 Date: May 3, 2022

(30) Foreign Application Priority Data
Nov. 4, 2019 (AT) A 50942/2019

(51) Int. Cl. C12M 1/26; A61L 27/24; A61L 27/50

(52) U.S. Cl. CPC C12M 33/12; A61L 27/24; A61L 27/50; A61L 2430/06; A61L 2430/10; A61L 2430/38

(57) ABSTRACT

The present invention relates to a method for producing compressed collagen comprising the steps of
a) pressing a gelled collagen in a first pressing direction; and
b) pressing the gelled collagen of step a) in a second pressing direction substantially orthogonal to the first pressing direction,
wherein the pressing in step a) is carried out at a pressure in the range of 0.01 to 0.05 bar and in step b) at a pressure in the range of 0.5 bar to 2 bar.

### TECHNICAL FIELD

The invention relates to compressed gelled collagen which can be used, inter alia, as an implant for the surgical treatment of a herniated disc.

### BACKGROUND OF THE INVENTION

Wear of joints in animals, particularly mammals, and humans is a common problem. The causes of this wear can be many and varied and can be due to increased stress or overexertion, such as from obesity or improper posture, pathological causes, low physical activity, or aging. In many cases, the damage to cartilage tissue, which serves as a shock absorber in the joints, caused by the above causes leads to increased wear of the joints. The consequences of wear on the joints include pain, which can also radiate to other parts of the body, and restricted mobility. One possible treatment for worn-out joints is surgery, accompanied by a joint replacement (i.e. endoprosthesis) or a cartilage transplantation or chondrocyte implantation.

A clinical picture in which cartilage tissue plays a decisive role is the herniated intervertebral disc. Intervertebral discs are pressure-elastic intervertebral discs located between the vertebral bodies of the cervical, thoracic and lumbar spine. The intervertebral disc is deformable and has limited compressibility and extensibility. Because of these properties, it participates in the movements of the spine. The intervertebral disc and the vertebral joints form a functional unit that reacts elastically even when subjected to strong mechanical stress.

Intervertebral discs consist of a gelatinous core (nucleus pulposus) on the inside. This tissue, which is poor in cells, consists of about 80% water and also of fibroblasts and type 2 collagen. The fibrocartilage tissue (annulus fibrosus) surrounds the nucleus pulposus. Hyaline cartilage is found at the upper and lower interfaces of the disc with the bones of the vertebral bodies.

Degenerative processes cause tears in the intervertebral disc with increasing age. After a subsequent pressure load, such as a jerky twisting of the spine, the nucleus pulposus passes through the annulus fibrosus, which is typically accompanied by sudden pain. This pathological process is called a herniated disc.

The escaped nucleus pulposus material can cause nerve irritation and/or bruising, which leads to pain, especially radiating into the legs or arms, or to sensory disturbances and loss of strength.

Current treatment options usually only eliminate the pain, but do not eliminate the cause itself. Herniated discs are treated both non-operatively, such as physiotherapy or painkillers, and surgically, such as a nucleotomy, in which the protruding part of the disc is removed. Following a nucleotomy, for example, a replacement material can be implanted.

WO 2012/004564 describes, inter alia, a method for producing a biomaterial obtained by the compression of collagen.

Patent application DE 100 26 789 A1 discloses a biomatrix as a cartilage substitute comprising at least 1.5 mg/mL of uncompressed collagen. An uncompressed collagen biomatrix is generally not suitable as a substitute for collagen subjected to high mechanical stress, such as nucleus pulposus, due to its lower strength.

Examples of a biomatrix that could be used as a substitute for nucleus pulposus material, among others, is disclosed in patent application DE 102 41 817 A1. The biomatrix described therein preferably uses gelled collagen fibres, which are subsequently compressed, whereby a final collagen concentration of up to 1000 mg/mL is possible. The compression is carried out, for example, by means of pressure which is applied in one dimension.

One-dimensional compression, as disclosed in DE 102 41 817 A1, results in a higher strength in the direction of compression compared to the non-compressed direction. However, this one-dimensional compressed material does not ensure sufficient strength for its use as a substitute for the nucleus pulposus, for example.

Therefore, it is an object of the present invention to provide a biomatrix comprising compressed collagen having high density and thus high strength for use as a replacement for the nucleus pulposus, for example, among others.

### SUMMARY OF THE INVENTION

It has been surprisingly found that compressed gelled collagen which has been pressed at least two-dimensionally has a higher strength than gelled collagen which has been pressed only one-dimensionally or not at all. Therefore, a first aspect of the present invention relates to a method for producing compressed collagen comprising the steps of
a) pressing a gelled collagen in a first pressing direction; and
b) pressing the gelled collagen of step a) in a second pressing direction substantially orthogonal to the first pressing direction,
wherein the pressing in step a) is carried out at a pressure in the range of 0.01 to 0.05 bar and in step b) at a pressure in the range of 0.5 bar to 2 bar.

In the process according to the invention, gelled collagen is first pressed in one direction. Subsequently, a second step takes place in which the one-dimensionally compressed collagen from the first process step is further pressed in a second pressing direction substantially orthogonal to the first pressing direction. In this second step, a compressed collagen matrix can be produced which has sufficient strength to be used, for example, as a nucleus pulposus replacement. Comparable strengths or collagen densities cannot be achieved with conventional methods (see, e.g., DE 102 41 817 A1).

Another aspect of the present invention relates to a collagen-containing product comprising compressed collagen obtainable by a process according to the present invention.

The compressed collagen obtainable by the process according to the present invention may be part of a collagen-containing product used for a wide variety of purposes.

A still further aspect of the present invention relates to an implant comprising a collagen-containing product according to the present invention or compressed collagen obtainable by a process according to the present invention.

Collagen, in particular compressed collagen, can be used, inter alia, as a cartilage substitute. Since cartilage is characterised above all by its strength combined with flexibility, it is decisive that a cartilage substitute has comparable properties, in particular also with regard to its biocompatibility, to endogenous cartilage. These properties can be achieved by pressing gelled collagen at least two-dimensionally. Therefore, a collagen-containing product comprising compressed collagen according to the invention is particularly suitable for use as an implant.

Another aspect of the present invention relates to a device for carrying out the method according to the invention, comprising:
a container comprising an inner space limited by a bottom and at least one wall, the container being adapted to receive a gelled water-containing collagen in the inner space; and
a piston which closes off the inner space of the container and is displaceable in a first pressing direction extending in the direction of the base,
characterised in that the at least one wall comprises segments, at least one of these segments being displaceable in a second pressing direction oriented substantially orthogonally to the first pressing direction.

A still further aspect of the present invention relates to a use of the device according to the invention in the process according to the invention.

### BRIEF DESCRIPTION OF FIGURES

FIG. 1 shows the results of determining flexibility when the implant according to the invention is applied to a portion of the human spine.

FIG. 2 shows the results of determining the pressure within the disc when using the implant according to the invention in a section of the human spine.

FIG. 3 shows the results of determining the height of the intervertebral disc when using the implant according to the invention in a section of the human spine.

FIG. 4 shows the results of determining the resistance to re-prolapse when using the implant according to the invention in a section of the human spine.

FIG. 5 shows an embodiment of the device according to the invention.

### DESCRIPTION OF THE EMBODIMENTS

In the method according to the invention, gelled collagen is first pressed in a first pressing direction and then in a second pressing direction. The pressing in the first and/or second pressing direction takes place at least once each. The starting material for this process is gelled collagen.

Gelled collagen can be produced by gelling dissolved collagen. The source of dissolved collagen may be cartilage, tendons, ligaments and bones, with cartilage being the most preferred source. The cartilage, tendons, ligaments and bones are preferably derived from mammals such as cattle, pigs, sheep and rats and are obtained by methods known in the prior art (e.g. DE 100 26 789). The use of certain methods for obtaining collagen, such as that of DE 100 26 789, have the advantage that the collagen does not have to be further processed after its extraction in order to be used in a mammal or human.

In addition to collagen from mammals, jellyfish and plants are also suitable as further sources of collagen.

“Compressed collagen”, as used herein, refers to gelled collagen that has a higher density than non-pressed collagen as a result of at least two pressing processes. Compressed collagen preferably has at least 10-fold, preferably at least 20-fold, even more preferably at least 40-fold compression (for example from 6 mg/ml to 240 mg/ml), even more preferably at least 80-fold compression, compared to the starting material (uncompressed collagen).

“A second pressing direction substantially orthogonal to a first pressing direction”, as used herein, means that in the method according to the invention at least one pressing step is provided having a pressing direction 9 substantially orthogonal to a first pressing direction 6.

“Pressing direction”, as used herein, is the direction in which pressure is applied to the gelled collagen.

The pressing of the gelled collagen in step a) in a first direction and/or the pressing in step b) in a second direction, which is substantially orthogonal to the first pressing direction, may be performed taking into account various parameters selected from a group consisting of pressure, duration, temperature and final dimension, preferably the parameters of pressure, duration and temperature may be varied to produce the compressed collagen according to the invention.

According to a preferred embodiment, the pressing in step a) is performed at a pressure in the range of 0.012 to 0.03 bar, preferably in the range of 0.015 to 0.025 bar, particularly preferably at 0.01833 bar, and/or in step b) at a pressure in the range of 1 to 1.5 bar, preferably at 1.01551 bar. These pressures do not include the air pressure. I.e. the actual pressure applied to the collagen to be compressed is the sum of the above mentioned pressures or pressure ranges and the prevailing air pressure (e.g. at sea level 1.01325 bar).

It has been shown that applying a pressure in the above-mentioned range to collagen leads to a gentle and at the same time uniform compression of the collagen. The pressure applied to the collagen is preferably applied to substantially all of the collagen to be compressed in a uniformly distributed manner.

According to a preferred embodiment, the pressing in step a) and/or step b) is performed by a linearly or stepwise increasing pressure.

The pressure applied to the collagen during pressing can either be constant (i.e. a defined pressure during steps a) and b)) or increase over time. The increase in pressure on the collagen can be linear or gradual. Of course, it is also possible to first apply an increasing pressure followed by a constant pressure or vice versa to the collagen to be compressed. With an increasing pressure, the end point (i.e. the maximum pressure to be reached) lies in the above-mentioned range.

According to a preferred embodiment, step a) is carried out for 1 to 48 hours, preferably for 6 to 24 hours, even more preferably for 8 hours to 18 hours, even more preferably for 10 hours to 14 hours, in particular for 12 hours, and/or step b) for 6 to 96 hours, preferably for 12 to 72 hours, even more preferably for 24 to 66 hours, even more preferably for 30 to 60 hours, even more preferably for 36 to 54 hours, even more preferably for 42 hours to 52 hours, even more preferably for 46 hours to 50 hours, in particular for 48 hours.

Due to the preferred compression pressures used in the process according to the invention, the pressure is preferably applied for a longer period of time, in particular in order to achieve the maximum possible compression in the compressed collagen body on the one hand and a constant compression in the collagen body on the other hand.

According to a preferred embodiment, step a) and/or step b) is carried out at a temperature of from 0° C. to 20° C., preferably from 0° C. to 15° C., even more preferably from 0° C. to 10° C., even more preferably from 1° C. to 9° C., even more preferably from 2° C. to 8° C., in particular from 4° C. to 6° C.

The pressed collagen produced by the process according to the invention may have different dimensions depending on the subsequent field of application. For certain applications (e.g. as a substitute for discus intervertebralis), according to a further preferred embodiment of the present invention, step a) and/or step b) of the process according to the invention results in a final dimension of the pressed gelled collagen of 1.5 to 5 mm, preferably of 2 to 4 mm, even more preferably of 2.4 mm to 3.0 mm, even more preferably of 2.6 mm to 2.8 mm, in particular of 2.7 mm edge length.

According to a preferred embodiment, the gelled collagen used for the two-dimensionally pressed gelled collagen can be provided by gelling a collagen-containing solution. The collagen-containing solution preferably comprises 1 to 20 mg, even more preferably 1 to 10 mg, even more preferably 2 mg to 8 mg, even more preferably 4 mg to 6 mg, in particular 5.5 mg to 6.5 mg, of dissolved collagen per mL of solution.

According to another preferred embodiment of the present invention, the collagen-containing solution is prepared by dissolving collagen in a first aqueous solution comprising 0.01 to 1%, preferably 0.05 to 0.5%, preferably 0, 01 to 0.2%, in particular 0.1%, of an organic acid having a pKS value of preferably 4.5 to 5, more preferably 4.6 to 4.9, in particular 4.7 to 4.8, wherein the organic acid is preferably acetic acid. Processes for the preparation of the collagen-containing solution are sufficiently known to the skilled person.

As mentioned at the outset, cartilage, tendons, ligaments and bones may serve as a source of dissolved collagen. Particularly preferably, the dissolved collagen is isolated from tendons of rat tails, preferably using the method described in DE 100 26 789 for its isolation.

According to a more preferred embodiment, the dissolved collagen is of collagen type 1. This collagen type is fibre-forming and is found in skin, tendons, bone, dentin, fibrocartilage and the cornea.

According to a further preferred embodiment, the dissolved collagen has a purity of greater than 90%, preferably greater than 95%, even more preferably greater than 99%, in particular greater than 99.8%.

According to a preferred embodiment of the present invention, the gelation of the dissolved collagen may be performed by adding a pH increasing agent. Preferably, the gelling process takes place over a period of time from 30 minutes to 10 hours, preferably from 45 minutes to 5 hours, more preferably from 1 to 4 hours, even more preferably from 2 to 3 hours, most preferably for about 2.5 hours. Particularly advantageous is the gelling of the collagen at a temperature in the range of from 15° C. to 50° C., preferably from 20° C. to 45° C., still more preferably from 25° C. to 40° C., still more preferably from 30° C. to 39° C., most preferably at about 34° C. During the gelling process, the solution may be stirred or mixed continuously or irregularly for improved mixing. It is crucial in this process that the mixing is done in such a way that the structure of the gelled collagen is not destroyed.

According to another more preferred embodiment of the present invention, the pH increasing agent is added in a ratio to the collagen in the collagen-containing solution of 0.1:2 (v/v), preferably 0.5:1.5 (v/v), more preferably 0.8:1.2 (v/v), most preferably 1:1 (v/v).

According to an even more preferred embodiment, the pH increasing agent is added to the collagen-containing solution in an amount such that the pH of the resulting collagen-containing solution is increased to from 7.0 to 9.0, preferably from 7.0 to 8.0, more preferably from 7.1 to 7.8, even more preferably from 7.3 to 7.5. The pH increasing agent is preferably added to the collagen-containing solution in an amount such that the solution in which collagen gels has a pH of from 7.0 to 7.4.

According to another more preferred embodiment of the present invention, the pH increasing agent is a buffering substance, preferably 2-(4-(2-hydroxyethyl)-1-piperazinyl)-ethanesulfonic acid (HEPES).

The pH increasing agent can either be dissolved in water or added directly (also as a solid substance) to the dissolved collagen.

According to a still more preferred embodiment, the pH increasing agent is part of an aqueous composition preferably comprising at least one component selected from the group consisting of Ham's F12 medium, sodium hydrogen carbonate and D-glucose.

In a preferred embodiment, following steps a) and b), the compressed collagen is transferred to a receiving vessel containing a second aqueous solution.

Preferably, the shape of the receiving vessel is not angular, but substantially elliptical, substantially round or circular.

Preferably, the second aqueous solution is a saline solution, more preferably PBS⁺.

The transfer into the receiving vessel preferably serves to adapt the outer shape of the compressed collagen to the cross-section of the receiving vessel by transitioning from a substantially angular cross-section to a substantially non-angular, more preferably substantially elliptical, still more preferably substantially round, most preferably substantially circular cross-section. This is preferably done by swelling of the collagen, more preferably by water absorption.

Another aspect of the present invention relates to a collagen-containing product comprising the compressed collagen according to the invention, which is obtainable by the process according to the invention.

According to a preferred embodiment, the product obtainable by the process according to the invention has an opaque to yellowish colour.

According to a preferred embodiment, the product has a three-dimensional shape, which is preferably substantially cylindrical. This cylindrical three-dimensional shape is preferably characterised in that it comprises a substantially non-angular, more preferably substantially elliptical, even more preferably substantially round, most preferably substantially circular base having a diameter of from 2 mm to 5 mm, preferably from 2.7 mm to 4.5 mm, even more preferably from 3.2 mm to 3.8 mm, in particular of about 3.5 mm.

The product according to the invention preferably exhibits visco-elastic and/or osmotic properties (preferably comparable to those of a native nucleus pulposus material), is mechanically stable and/or highly compressed (preferably up to or more than 240 mg/ml).

The compressed collagen produced according to the invention also exhibits the properties of a hydrogel.

Another aspect of the present invention relates to an implant comprising the collagen-containing product according to the invention.

The implant according to the invention can be used for a wide variety of purposes. The implant according to the invention can be used as a nucleus pulposus replacement, meniscus replacement, ligament replacement and/or tendon replacement, whereby it is particularly suitable as a nucleus pulposus replacement, for the treatment of meniscus injuries (e.g. meniscus tear) and ligament injuries (carpal ligament, yellow ligament, inguinal ligament, patellar ligament, anterior cruciate ligament, posterior cruciate ligament). Depending on the field of application, the collagen-containing product according to the invention can be formed into a suitable implant and implanted.

According to a further preferred embodiment, the implant is a long-term implant, which is characterised in that cells, even more preferably cells of the intervertebral disc, can migrate. Through the migration of the cells, the implant according to the invention, in the case of an intervertebral disc implant, can become a tissue similar to a nucleus pulposus.

According to a further preferred embodiment, the implant can be implanted by means of an operation. This operation can be performed on animals, in particular mammals, and humans.

In the course of the operation, the implant according to the invention can be displaced from the receiving vessel to the target site, which is preferably located inside the annulus fibrosus, by means of an auxiliary means. Due to its properties, the implant according to the invention is preferably suitable as a replacement of nucleus pulposus material.

According to a more preferred embodiment, the surgery is performed using minimally invasive surgical techniques.

The surgery is preferably performed as a treatment for a herniated disc, which is preferably associated with pain, more preferably back and/or leg pain.

The amount of the implant according to the invention is preferably determined according to the height of the adjacent intervertebral disc and/or pressed into its target location.

The operation preferably takes place following a nucleotomy and/or is preferably completed by closure of the annulus fibrosus.

According to a preferred embodiment, the collagen-containing product or implant comprises compressed collagen having a density greater than 150 mg/mL, more preferably greater than 200 mg/mL, most preferably greater than 240 mg/mL.

Preferably, the collagen-containing product and/or the implant having the above density is prepared by the process according to the invention.

Another aspect of the present invention relates to a device 1 for carrying out the process according to the invention, comprising:
a container 2 comprising an inner space 5 limited by a bottom 3 and at least one wall 4, the container 2 being adapted to receive a gelled water-containing collagen in the inner space 5, and
a piston 7 which closes off the inner space 5 of the container 2 and is displaceable in a first pressing direction 6 extending in the direction of the base,
wherein the at least one wall comprises 4 segments, wherein at least one of these segments 8 is displaceable in a second pressing direction 9 oriented substantially orthogonally to the first pressing direction 6.

The container 2 preferably comprises a sterilisable cell culture suitable material, preferably Polytetraflon® HS 11097, or is at least partially coated with this cell culture suitable material on the surface which is brought into contact with the collagen.

According to a preferred embodiment of the present invention, the bottom 3, the at least one wall 4 and/or the plunger 7 comprises at least one opening 10 opening into the inner space 5 for removing water from the inner space 5 of the container 2.

This at least one opening 10 is preferably designed in such a way that gelled collagen cannot be pressed through this at least one opening 10 in the course of a pressing process. Therefore, the at least one opening 10 preferably has a size of from 50 μm to 500 μm, preferably from 100 to 300 μm, even more preferably from 120 to 250 μm, even more preferably from 130 to 200 μm, in particular from 160 μm.

The base 3, the at least one wall 4 and/or the plunger 7 may also comprise or consist of a sufficiently mechanically stable material, which may be porous, preferably over the entire area in contact with the collagen when properly used, and water-permeable. The use of porous and water-permeable materials is particularly advantageous, as this can be produced without the insertion of separate openings (e.g. by drilling holes). The pores of a porous material correspond to the openings 10 as defined herein.

Particularly suitable are porous plastics which are composed of granules which are bonded together in a sintering process. This creates cavities (pores) that allow gases and especially liquids such as water to flow through the parts. Porous plastic products, applicable in the device according to the invention, can be made from various thermoplastic materials, whereby ultra-high molecular weight polyethylene (UHMWPE), high-density polyethylene (HDPE), polypropylene (PP), polytetrafluoroethylene (PTFE), polyvinylidene fluoride (PVDF), polyethersulfone (PES) and PE/PP copolymer are particularly preferred. The pore size is dimensioned such that water can penetrate the material, but not the collagen-containing material.

The term “water” as used herein may include other substances and salts present in the starting material to be compressed. In this context, “water” may be equated with “aqueous solution”.

According to another preferred embodiment of the present invention, the at least one opening 10 for removing water from the inner space 5 of the container 2 is connected to at least one further container 11 for receiving water.

In order to remove the compression water produced during the compression of the collagen-containing material, the device according to the invention comprises an additional container 11 for water absorption. This prevents water from escaping from the device directly into the environment. After completion of the compression process, the water can be removed from the container 11.

According to a further preferred embodiment of the present invention, the device 1 comprises at least one locking means 12, which is designed to lock the piston 7, which is displaceable in the first pressing direction 6, and/or the segment 8, which is displaceable in the second pressing direction 9, in an end position of the first 6 and/or second pressing direction 9 on the container 2.

A locking device is particularly advantageous, as it enables the piston 7, which is displaceable in the first pressing direction 6, and/or the segment 8, which is displaceable in the second pressing direction 9, to be fixed in one position. If, after the compression process, the pressure on the collagen to be compressed is reduced or removed completely, the shape of the compressed collagen can be maintained by means of a locking device. In addition, the locking device allows the collagen to be compressed in a second direction. Thus, it is possible to compress the collagen in both compression directions in a single device without using a second device.

The locking of the at least one locking means 12 is preferably releasable.

The at least one locking means 12 is preferably a screw connection (e.g. screw, nut), a nail, a pin or a clamp.

Another aspect of the present invention relates to a use of the device 1 according to the invention in the method according to the invention.

According to a preferred embodiment of the present invention, steps a) and/or b) are carried out in the container 2 of the device 1 according to the invention.

Since water is not compressible, it is advantageous when pressing the gelled collagen if the container 2 in which the gelled collagen is pressed is designed to be at least partially permeable to water.

The bottom 3, the at least one wall 4 and/or the plunger 7 preferably comprise at least one opening 10 opening into the inner space 5 for removing water from the inner space 5 of the container 2. These openings 10 are preferably designed in such a way that gelled collagen is not pressed through these openings 10 in the course of a pressing process. Therefore, the walls or boundaries have openings 10 in a size of from 50 μm to 500 μm, preferably from 100 to 300 μm, even more preferably from 120 to 250 μm, even more preferably from 130 to 200 μm, in particular from 160 μm.

According to a preferred embodiment of the present invention, the gelling of the dissolved collagen is carried out in the same container 2 as the pressing of the gelled collagen. This container 2 preferably consists of a sterilisable material suitable for cell culture, preferably Polytetraflon® HS 11097, or is at least partially coated with this material suitable for cell culture on the surface which is brought into contact with the collagen.

### DETAILED DESCRIPTION OF THE FIGURES

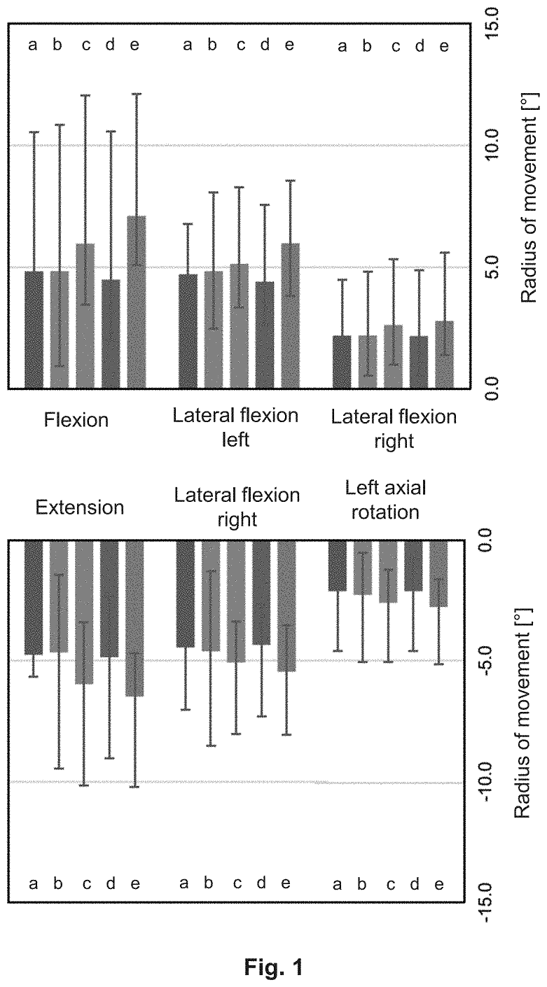

FIGS. 1 to 4 are described in the section of the examples.

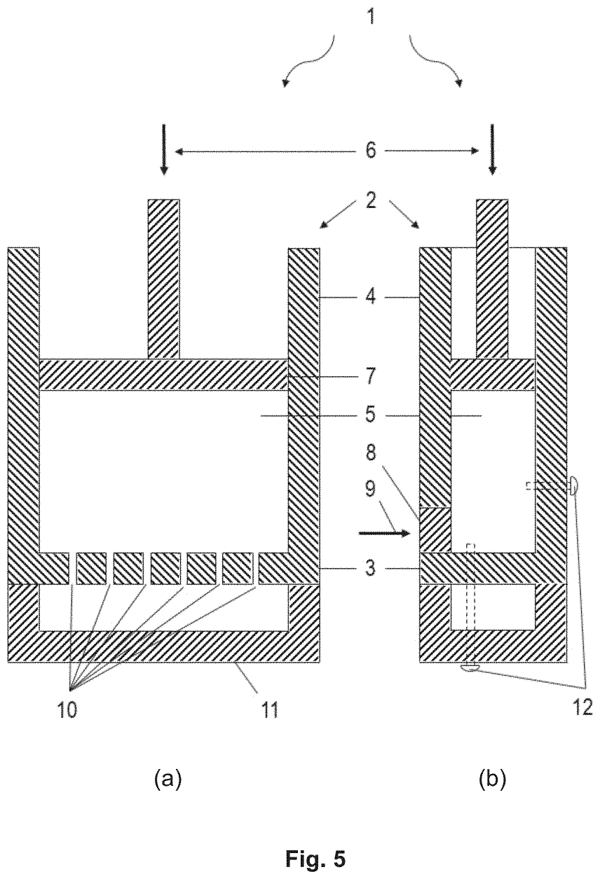

FIG. 5 shows a section of the front view (a) and a section of the side view (b), independent of the first section, of a preferred embodiment of the device 1 according to the invention, which comprises a receptacle 2 comprising an inner space 5 delimited by a bottom 3 and at least one wall 4. The container 2 is designed to receive a gelled water-containing collagen in the inner space 5. Furthermore, the container 2 comprises a piston 7 which closes off the inner space 5 of the container 2 and is displaceable in a first pressing direction 6 extending in the direction of the base. The device 1 is characterised in that the at least one wall 4 comprises segments, at least one of these segments 8 being displaceable in a second pressing direction 9 oriented substantially orthogonally to the first pressing direction 6. The base 3 has an opening 10 opening into the inner space 5 for removing water from the inner space 5 of the container 2. This opening 10 for removing water from the inner space 5 of the container 2 is connected to a further container 11 for receiving water. The device 1 comprises two locking means 12, which are designed to lock the piston 7, which can be displaced in the first pressing direction 6, and the segment 8, which can be displaced in the second pressing direction 9, in an end position of the first and second pressing directions on the container 2.

Examples illustrating the invention are described below.

### EXAMPLES

### Example 1: Preparation of Collagen GC

Example 1 describes the preparation of the collagen from rat tail tendons and refers to commercially available collagen type I solutions.

The isolation of collagen from rat tail tendons can be carried out as described in the prior art, for example in DE 100 26 789. In this case, collagen MS isolated from older rats is obtained as collagen GC.

### Example 2: Gelation of Collagen GC

Example 2 describes the gelation of collagen GC.

The gelation of collagen GC included, by way of example, the following steps:
a. Preparation of a gel neutralisation solution comprising the following ingredients, shown in Table 1:

TABLE 1
Ingredients of the gel neutralisation solution.
Ingredient

D-glucose
Ham's F-12 medium (10x, with stable glutamin, without sodium hydrogen
carbonate)
HEPES solution (3M, pH 7.8)
Sodium hydrogen carbonate
dH₂O

b. Mixing of the collagen GC solution with the gel neutralisation solution in a ratio of 1:1 (v/v) until homogeneity, resulting in neutralisation to physiological pH.
c. Pouring the homogeneous solution from the previous step into a pouring chamber of rectangular cross-section (length: 20 cm, width: 2.7 cm, height 5 cm). The pouring chamber is placed in an apparatus.
d. Gelation of the homogeneous solution of the previous step in the casting chamber for 2.5 h at 34° C.

### Example 3: Pressing the Gelled Collagen GC

Example 3 describes the two-dimensional pressing of the gelled collagen GC.

The two-dimensional pressing of the gelled collagen GC included the following exemplary steps:
a. Modification of the apparatus of Example 2, wherein the plastic cover was replaced by a cover bonnet with a punch (weight bonnet+punch 1.01 kg).
b. Moistening of the lower filter plate with PBS⁺ solution
c. Use of the spacers
d. Closing the apparatus with the cover bonnet
e. First pressing for 12 h at 2-6° C. to a final dimension of 2.7 mm
f. Removal of the one-dimensionally pressed gelled collagen GC
g. Insertion of the one-dimensionally pressed gelled collagen GC into the apparatus while opening the casting chamber sideways.
h. Use of the spacers
i. Second pressing at 2-6° C. to a final size of 2.7 mm comprised the following steps:
   i. 2.5 kg for 4 h
   ii. 5 kg for 20 h
   iii. 10 kg for 24 h

### Example 4: Further Processing of the Pressed Collagen GC

Example 4 describes the further processing of the two-dimensional pressed collagen GC.

The further processing of the two-dimensionally pressed collagen GC included the following exemplary steps:
a. Removal of the two-dimensionally pressed collagen GC with an edge length of 2.7 mm from the apparatus
b. Confectioning the two-dimensionally pressed collagen GC into, for example, 5 cm long pieces
c. Transfer the pieces from the previous step into application tubes filled with PBS⁺ buffer with an inner diameter of 3.5 mm, whereby the pieces swell to a round cross-section with a diameter of 3.5 mm.
d. Storage of the pieces from the previous step at 2-10° C.
e. Gamma sterilisation of the pieces from either of the two previous steps at 15-20 kGy, preferably 17.5-17.7 kGy at 2-37° C.

### Example 5: Biomechanical Tests of the Product of the Invention as an Implant after a Herniated Disc

Example 5 describes the biomechanical properties of the product according to the invention, which was used as an implant in parts of human spines after a herniated disc.

Due to the properties of the product according to the invention, it can be used as an implant in the treatment of a herniated disc. These properties include, for example, the collagen content of 240 mg/mL, which is similar to that of native nucleus pulposus.

### Example 5a: Fabrication of the Implant

The implant according to the invention was manufactured according to the method of the invention. The implant consisted of 98.5% pure native Kollagen type I and had the following dimensions: 50 mm×3.5 mm (length×diameter).

### Example 5b: Origin and Preparation of Spine Samples

The in vitro experiments were performed on six samples representing functional units of the human lenticular spine. Table 2 provides an overview of the samples.

TABLE 2

| | | | Intervertebral |
|---|---|---|---|
| Sample | Intervertebral | Pfirmann | disc height |
| number | disc level | grade | [mm] |
| 1 | L5-S1 | 3 | 7.4 |
| 2 | L3-L4 | 2 | 10.4 |
| 3 | L5-S1 | 3 | 8.5 |
| 4 | L2-L3 | 2 | 7.6 |
| 5 | L4-L5 | 3 | 7.2 |
| 6 | L3-L4 | 2 | 8.5 |

The six samples were from four human donors. Each sample consisted of an intervertebral disc and its two adjacent vertebrae, resulting in the disc level. A Pfirmann grade of 1-3 was considered adequate disc quality for the indication of disc herniation.

Soft tissue and muscle fibres were removed from the samples. In order to be able to mount the specimens adequately in the test devices, they were embedded in polymethylmethacrylate (PMMA) at both the cranial and caudal ends.

### Example 5c: Sample Handling Steps

Creation of a Posterior Defect

Using bone forceps, each sample was subjected to a laminectomy. A “box-cut” of 6×7 mm was created in the annulus fibrosus. A box-cut is a rectangular opening that can be created using a gouge. This cavity was created in order to be able to close the annulus fibrosus with an appropriate implant in a later step.

Creation of a Disc Prolapse

By means of a cyclic stress test, a disc prolapse, i.e. the passage of the nucleus pulposus material through the annulus fibrosus, was generated.

The cyclic loading test was performed in the servohydraulic loading frame (Instron 8871, Darmstadt, Germany). A custom-built rotary base was mounted on the support surface of the material testing machine. The sample was flanged to the rotary base, which maintained a rotational speed of 360°/min. The rotation base was then shifted sideways by 30 mm to achieve an eccentric cyclic loading of the specimens. The load on the hydraulic piston was increased linearly to 350 N. A sinusoidal force in the range of 100-600 N at 5 Hz was then applied up to a maximum of 100,000 cycles. This maximum of 100,000 cycles was defined in order to perform the experiments within 12 hours and thus avoid degradation of the samples. The 30 mm lateral displacement effectively acted as a lever arm so that a maximum of 18 Nm could be applied. The test or loading was stopped when the nucleus pulposus material leaked out. The leaked nucleus pulposus material was removed, including from the generated box-cut. A partial nucleotomy was therefore performed.

Implantation of the implant of the invention
The implantation was minimally invasive. The implant according to the invention was pressed into the disc by means of a punch. The nucleus pulposus material is enclosed by the annulus fibrosus in an intact intervertebral disc and thus receives natural support. In order to determine the functionality of the implant according to the invention and to compare it with the nucleus pulposus material in an intact intervertebral disc, the defect created by means of a “box-cut” was closed by the annulus fibrosus closure device Barricaid® (Intrinsic Therapeutics, Germany).

### Example 5d: Performance of the Biomechanical Tests

Test Environment

The tests were carried out at room temperature. The samples were wrapped in gauze soaked in saline solution to prevent dehydration and de-integration.

Determination of Flexibility

This test was performed with the disc intact, after box-cut, after nucleotomy, after implantation of the implant of the invention and after the cyclic loading test.

To determine flexibility, the specimen was first fixed at the caudal end. Pure flexion moments were applied to the cranial end at a constant rate of 1.5°/s. The flexion moments were applied at a rate of ±7.5 Nm. Here ±7.5 Nm were used for the following directions: lateral flexion right/left (+/−), flexion/extension (+/−) and left/right (+/−) axial rotation.

The samples were subjected to 3.5 loading cycles. The first 2.5 cycles served as pre-cycles to minimise the effect of a visco-elastic response. The last cycle was used for the results.

Determination of the Pressure Inside the Disc

This test was performed with the disc intact, after box-cut, after nucleotomy, after implantation of the implant of the invention and after the cyclic loading test.

The pressure within the disc was recorded by means of an implanted pressure sensor positioned in the nucleus of the disc.

Determination of Disc Height

This test was performed with the disc intact, after box-cut, after disc herniation, after nucleotomy, after implantation of the inventive implant and after the cyclic loading test.

The height of the disc was determined using the Instron material testing machine. A pre-load of 100 N was applied to the specimen for 5 s before the measurement.

Cyclic Load Test

This test was performed after implantation of the implant according to the invention.

The cyclic loading tests were performed in the servohydraulic loading frame (Instron 8871, Darmstadt, Germany). A custom-built rotary base was mounted on the support surface of the materials testing machine. The samples were flanged to the rotary base, which maintained a rotational speed of 360°/min. The rotation base was then shifted sideways by 30 mm to achieve an eccentric cyclic loading of the specimens. The load on the hydraulic piston was increased linearly to 350 N. A sinusoidal force in the range of 100-600 N at 5 Hz up to a maximum of 100,000 cycles was then applied. The 30 mm lateral displacement effectively acted as a lever arm so that a maximum of 18 Nm could be applied. After implantation, the test was not stopped before 100,000 loading cycles at the exit of core material, but at the exit of the implant. This maximum of 100,000 cycles was defined in order to perform the experiments within 12 hours and thus avoid degradation of the samples.

Determination of the Resistance to Re-Prolapse

This test was performed after the cyclic loading test.

For macroscopic assessment, the discs were cut at the mid-transverse plane of the disc. Photographs were taken with a digital camera to determine the condition of the implant.

### Example 5e: Results of the Biomechanical Tests

Determination of Flexibility

FIG. 1 shows the results of this test, which was carried out on the intact disc (a), after the “box-cut” (b), after the nucleotomy (c), after the implantation of the implant of the invention (d) and after the cyclic loading test (e).

It can be seen that the range of motion after implantation decreased to a comparable level with that of the intact disc. This positive effect was reversed by the cyclic loading test.

Determination of the Pressure Inside the Disc

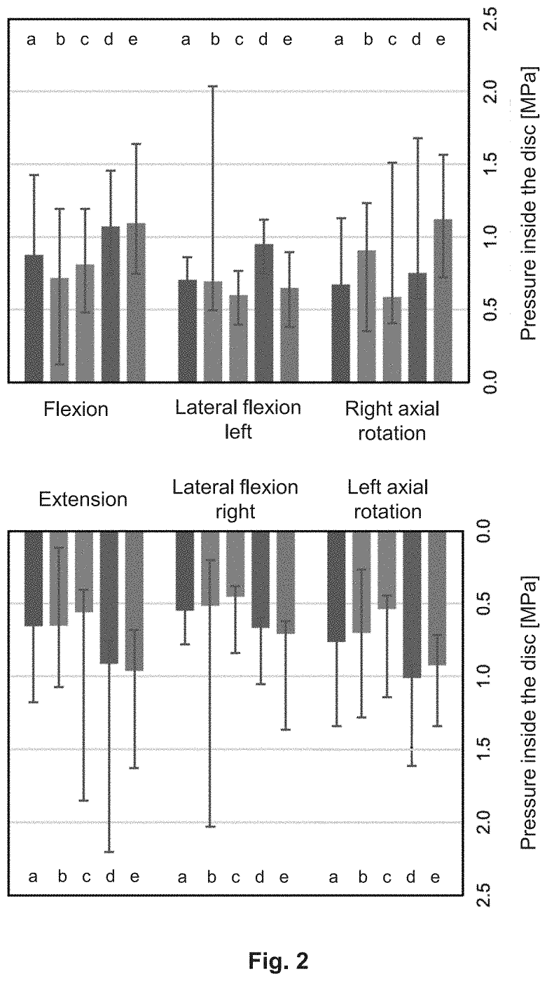

FIG. 2 shows the results of this test, which was carried out on the intact disc (a), after the box-cut (b), after the nucleotomy (c), after the implantation of the implant of the invention (d) and after the cyclic loading test (e).

The pressure within the intervertebral disc could be increased by the implantation even above that in the intact intervertebral disc. In most cases, the pressure could not be reduced even by the cyclic load test.

Determination of the Disc Height

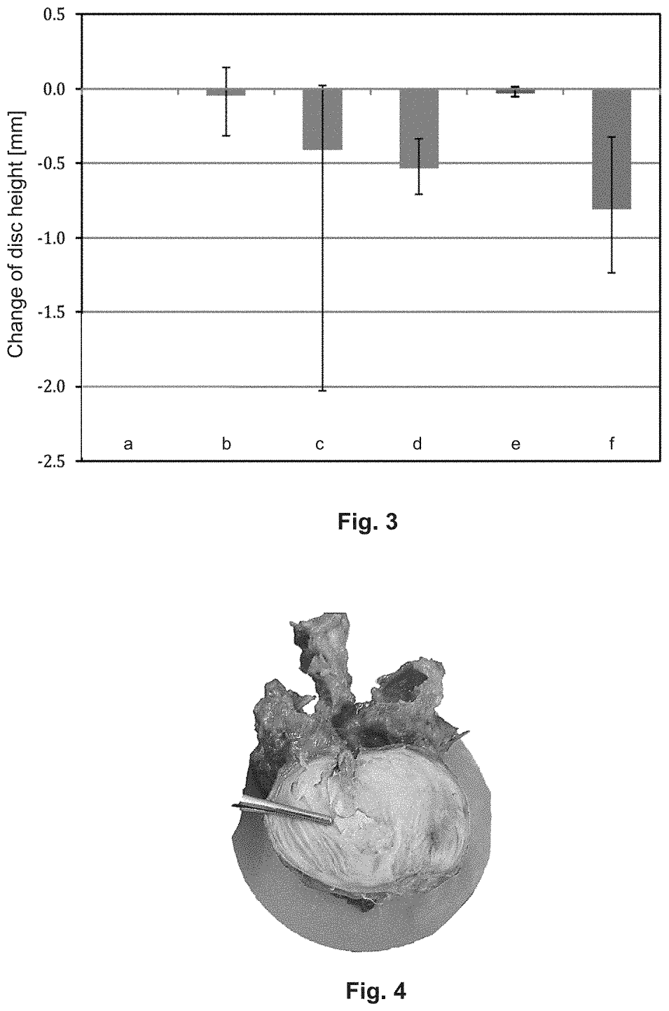

FIG. 3 shows the results of this test, which was carried out on the intact intervertebral disc (a), after the “box-cut” (b), after the herniated disc (c), after the nucleotomy (d), after the implantation of the implant of the invention (e) and after the cyclic loading test (f.)

The height of the disc could be lowered to that of an intact disc by implantation. Again, the effect was reversed by the cyclic loading test.

Determination of the Resistance to a Renewed Prolapse

FIG. 4 shows the result of this test. The forceps point to the implant. The implant remained within the surrounding nucleus pulposus material at the implanted site. No new disc prolapse occurred as a result of the cyclic loading test.

Summary of Results

An intervertebral disc prolapse was added to a functional section of the human lumbar spine. The implant according to the invention was able to adapt both the flexibility and the height of the intervertebral disc to those of the intact intervertebral disc. Following implantation, the pressure within the intervertebral disc even exceeded that of the intact intervertebral disc.

Possibility of Using the Implant of the Invention in Humans

As shown by the above examples, the implant according to the invention is suitable for restoring the properties of an intervertebral disc after a herniated disc. In general, a herniated disc associated with back and/or leg pain can be treated by means of a nucleotomy. In the course of this, nucleus pulposus material is removed. The implant according to the product of the invention can be used as a replacement for the removed nucleus pulposus material and can be pushed into the defect by means of plungers from the application tube. The amount of implanted implant can be determined according to the height of the adjacent discs, as described for example in Hong et al. (Asian Spine Journal, Vol. 4, No. 1, pp. 1-6, 2010).

The use of the implant according to the invention as a replacement for the removed nucleus pulposus material can be carried out by means of a minimally invasive surgical technique.

Following the insertion of the implant, the annulus fibrosus should be closed. Suitable closure systems are commercially available, such as Barricaid® (Intrinsic Therapeutics, Germany).

1. A method of producing compressed collagen comprising:
a) pressing a gelled collagen in a first pressing direction and
b) then pressing the gelled collagen in a second pressing direction substantially orthogonal to the first pressing direction,
wherein the pressing in the first pressing direction is carried out at a first pressure in the range of 0.01 to 0.05 bar, and in the pressing in the second pressing direction is carried out at a second pressure in the range of 0.5 bar to 2 bar, to produce the compressed collagen.

2. The method according to claim 1, wherein the first pressure ranges from 0.012 to 0.03 bar, and/or the second pressure ranges from 1 bar to 1.5 bar.

3. The method according to claim 1, wherein the pressing in the first pressing direction, the pressing in the second pressing direction, or both, is carried out by linearly or stepwise increasing pressure.

4. The method according to claim 1, wherein the pressing in the first pressing direction is carried out for 1 to 48 hours, and/or the pressing in the second pressing direction is carried out for 6 to 96 hours.

5. The method according to claim 1, the gelled collagen is provided by gelling a collagen-containing solution comprising 1 to 20 mg of dissolved collagen per mL of the collagen-containing solution.

6. The method according to claim 5, wherein the collagen-containing solution is prepared by dissolving collagen in an aqueous solution comprising 0.01 to 1% of an organic acid having a pKS value of 4.5 to 5.

7. The method according to claim 5 wherein the gelling is effected by adding a pH-increasing agent to the collagen-containing solution.

8. The method according to claim 7, wherein the pH-increasing agent is a buffer substance comprising 2-(4-(2-hydroxyethyl)-1-piperazinyl)-ethanesulfonic acid (HEPES).

9. The method according to claim 1, further comprising transferring the compressed collagen into a receiving vessel containing a second aqueous solution.

10. The method according to claim 9, wherein the second aqueous solution is a salt-containing solution.

11. A collagen-containing product comprising the compressed collagen obtained by the method according to claim 1.

12. An implant comprising the collagen-containing product according to claim 11.

13. The implant according to claim 12, wherein the implant is suitable as a nucleus pulposus replacement, a meniscus replacement, a ligament replacement and/or a tendon replacement.

14. A device for producing compressed collagen, comprising:
a) a container comprising an inner space delimited by a bottom and at least one wall, the container being designed adapted to receive a gelled water-containing collagen in the inner space, and
b) a piston which closes off the interior inner space of the container and is displaceable in a first pressing direction extending in the direction of the base,
wherein the at least one wall comprises at least one segment being displaceable in a second pressing direction oriented substantially orthogonally to the first pressing direction.

15. The device according to claim 14, wherein the base, the at least one wall and/or the piston has at least one opening for removing water from the inner space of the container.

16. The device according to claim 15, wherein the at least one opening for removing water from the inner space of the container is connected to at least one further container for receiving water.

17. The device according to claim 14, wherein the device comprises at least one locking means for locking the piston displaceable in the first pressing direction and/or the at least one segment displaceable in the second pressing direction in an end position of the first pressing direction and/or the second pressing direction on the container.

18. A method of producing compressed collagen comprising, in the device of claim 14,
a) pressing a gelled collagen in the first pressing direction with the piston, and
b) then pressing the gelled collagen in the second pressing direction with the at least one segment,
thereby producing the compressed collagen.

19. The collagen-containing product of claim 11, wherein the compressed collagen has a density greater than 150 mg/mL.

20. The implant of claim 12, wherein the compressed collagen has a density greater than 150 mg/mL.
